(19) United States
(12) Patent Application Publication
(10) Pub. No.: US 2023/0033914 A1
(43) Pub. Date: Feb. 2, 2023

(54) SYSTEM AND METHOD FOR PRESCRIBING DRUGS BASED ON A PATIENT'S INSURANCE PROVIDER

(71) Applicant: Byron Jones, Lakeside, CA (US)

(72) Inventor: Byron Jones, Lakeside, CA (US)

(21) Appl. No.: 17/851,839

(22) Filed: Jun. 28, 2022

Related U.S. Application Data
(60) Provisional application No. 63/225,988, filed on Jul. 27, 2021.

(51) Int. Cl. G16H 20/10

(52) U.S. Cl. CPC G16H 20/10

(57) ABSTRACT

A system for determining suitable medications for a patient based on the patient’s insurance provider is disclosed, including at least one user computing device in operable connection with a user network. An application server is in operable communication with the user network to host an application system for providing a plurality of medication information. The application system having a user interface for providing access to the application system through the user computing device. A plurality of tiles are displayed on the user interface, wherein each tile is associated with a medication.

### CROSS-REFERENCE TO RELATED APPLICATIONS

The present application claims priority to U.S. Provisional Application No. 63/225,988 filed Aug. 3, 2021, titled “SYSTEM AND METHOD FOR PRESCRIBING DRUGS BASED ON THE PATIENT’S INSURANCE PROVIDER”, which is hereby incorporated by reference in its entirety.

### TECHNICAL FIELD

The embodiments generally relate to computerized systems for determining a drug to prescribe based on various factors including a patient’s insurance provider, condition, presenting symptoms, and the like.

### BACKGROUND

Prescription drugs (i.e., prescription medications or prescription medicine) is a pharmaceutical drug that legally requires a prescription to be dispensed. In the United States, the federal government authorizes physicians, physician assistants, nurse practitioners, veterinarians, dentists, and optometrists to prescribe various controlled substances. Patients often use their insurance provider to at least partially pay for their prescribed medications. Over time, the financial systems and rules for providing healthcare and prescription benefits to a patient have become increasingly complex. For example, financial systems include rules for formulary tiers, deductible protocols, benefit parameters (e.g., maximum coverage costs, etc.), prior authorization limits, limitations, exemptions, and the like are all factors in determining if the patient is covered for the prescription of a particular drug or if they must pay in-part or in-full for the medication.

Prescribing physicians or other medical professionals often only prescribe a small number of medications, based on their specialty, clientele, or preferred cases. Of those medications, the medical professional may be aware of which medications (from a variety of available medications which are approved and available) are approved by a relatively high percentage of insurance providers. In such, the medical professional may favor prescribing that medication which is likely to be approved by the patient’s insurance, rather than providing a more suitable medication.

### SUMMARY OF THE INVENTION

This summary is provided to introduce a variety of concepts in a simplified form that is disclosed further in the detailed description of the embodiments. This summary is not intended to identify key or essential inventive concepts of the claimed subject matter, nor is it intended for determining the scope of the claimed subject matter.

A system for determining suitable medications for a patient based on the patient’s insurance provider is disclosed, including at least one user computing device in operable connection with a user network. An application server is in operable communication with the user network to host an application system for providing a plurality of medication information. The application system having a user interface for providing access to the application system through the user computing device. A plurality of tiles are displayed on the user interface, wherein each tile is associated with a medication.

### BRIEF DESCRIPTION OF THE DRAWINGS

A complete understanding of the present embodiments and the advantages and features thereof will be more readily understood by reference to the following detailed description when considered in conjunction with the accompanying drawings wherein:

FIG. 1 illustrates a block diagram of a computing system, according to some embodiments;

FIG. 2 illustrates a block diagram of a computing system and an application program, according to some embodiments;

FIG. 3 illustrates a flowchart of the process for creating an indication category, drug container based on the indication and assigning medications to the container, according to some embodiments;

FIG. 4 illustrates a flowchart of a process for generating a warning label, according to some embodiments;

FIG. 5 illustrates a flowchart of the process of generating a dosing schedule, according to some embodiments;

FIG. 6 illustrates a flowchart of the process of generating a dosing form, according to some embodiments;

FIG. 7 illustrates a flowchart of the process of generating a manufacturer table, according to some embodiments;

FIG. 8A illustrates a flowchart of the process of distributing medication information to the user, according to some embodiments;

FIG. 8B illustrates a flowchart of the process of distributing medication information to the user, according to some embodiments;

FIG. 9 illustrates a flowchart of the process of determining if the user is a prescriptive authority or if the user is licensed, according to some embodiments;

FIG. 10A illustrates a flowchart of the administrative processes performed via the administrative user utilizing the system, according to some embodiments;

FIG. 10B illustrates a flowchart of the administrative processes performed via the administrative user utilizing the system, according to some embodiments;

FIG. 11 illustrates a flowchart of a process of the user querying the system to receive medication information, according to some embodiments;

FIG. 12 illustrates a screenshot of the insurance plan selection interface, according to some embodiments;

FIG. 13 illustrates a screenshot of the plan customization interface, according to some embodiments;

FIG. 14 illustrates a screenshot of the medication selection interface, according to some embodiments;

FIG. 15 illustrates a screenshot of an organized interface showing various medications based on various parameters for each medication, according to some embodiments;

FIG. 16 illustrates a screenshot of the tiles also shown in FIG. 16, according to some embodiments; and

FIG. 17 illustrates a screenshot of the medication information interface, according to some embodiments.

### DETAILED DESCRIPTION

The specific details of the single embodiment or variety of embodiments described herein are to the described system and methods of use. Any specific details of the embodiments are used for demonstration purposes only, and no unnecessary limitations or inferences are to be understood thereon.

Before describing in detail exemplary embodiments, it is noted that the embodiments reside primarily in combinations of components and procedures related to the system. Accordingly, the system components have been represented, where appropriate, by conventional symbols in the drawings, showing only those specific details that are pertinent to understanding the embodiments of the present disclosure so as not to obscure the disclosure with details that will be readily apparent to those of ordinary skill in the art having the benefit of the description herein.

In this disclosure, the various embodiments may be a system, method, and/or computer program product at any possible technical detail level of integration. A computer program product can include, among other things, a computer-readable storage medium having computer-readable program instructions thereon for causing a processor to carry out aspects of the present disclosure.

The embodiments relate to a system and method for prescribing drugs based on the patient’s insurance provider. The system helps physicians determine the best drug to prescribe rather than simply prescribing a drug that the physician knows is accepted by a large percentage of insurance providers. In use, the physician utilizes a computing device interface to select a disease they are treating. The physician then selects an insurance provider who insures the patient. Each drug that is approved by the selected insurance provider is shown to the physician along with formulary data to help the physician make an informed decision. The physician will also be provided with the drug tier to aid in the determination of the copay amount. Further, the system includes the insurance provider requirements for providing the drug (i.e., is the drug easy or difficult to provide and what is needed in order for the insurance provider to cover the particular drug).

As used herein, the term “formulary” or “prescription formulary” refers to a list of medications that are approved to be prescribed to a particular patient.

As used herein, the term “medication” may refer to any medicaments, medicine, pharmaceutical drug, procedure, treatment plan, therapeutic element, etc. used to diagnose, cure, treat, prevent disease, or otherwise perform drug therapy.

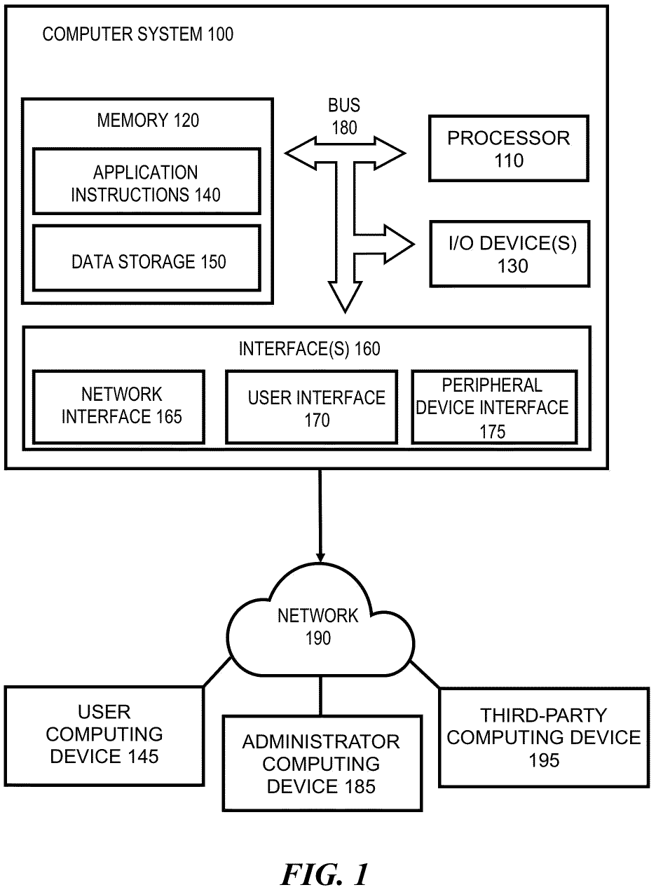

FIG. 1 illustrates an example of a computer system 100 that may be utilized to execute various procedures, including the processes described herein. The computer system 100 comprises a standalone computer or mobile computing device, a mainframe computer system, a workstation, a network computer, a desktop computer, a laptop, or the like. The computing device 100 can be embedded in another device, e.g., a mobile telephone, a personal digital assistant (PDA), a mobile audio or video player, a game console, a Global Positioning System (GPS) receiver, or a portable storage device (e.g., a universal serial bus (USB) flash drive).

In some embodiments, the computer system 100 includes one or more processors 110 coupled to a memory 120 through a system bus 180 that couples various system components, such as an input/output (I/O) devices 130, to the processors 110. The bus 180 may be any of several types of bus structures including a memory bus or memory controller, a peripheral bus, and a local bus using any of a variety of bus architectures. For example, such architectures include Industry Standard Architecture (ISA) bus, Micro Channel Architecture (MCA) bus, Enhanced ISA (EISA) bus, Video Electronics Standards Association (VESA) local bus, and Peripheral Component Interconnect (PCI) bus, also known as Mezzanine bus.

In some embodiments, the computer system 100 includes one or more input/output (I/O) devices 130, such as video device(s) (e.g., a camera), audio device(s), and display(s) are in operable communication with the computer system 100. In some embodiments, similar I/O devices 130 may be separate from the computer system 100 and may interact with one or more nodes of the computer system 100 through a wired or wireless connection, such as over a network interface.

Processors 110 suitable for the execution of computer readable program instructions include both general and special purpose microprocessors and any one or more processors of any digital computing device. For example, each processor 110 may be a single processing unit or a number of processing units and may include single or multiple computing units or multiple processing cores. The processor(s) 110 can be implemented as one or more microprocessors, microcomputers, microcontrollers, digital signal processors, central processing units, state machines, logic circuitries, and/or any devices that manipulate signals based on operational instructions. For example, the processor(s) 110 may be one or more hardware processors and/or logic circuits of any suitable type specifically programmed or configured to execute the algorithms and processes described herein. The processor(s) 110 can be configured to fetch and execute computer readable program instructions stored in the computer-readable media, which can program the processor(s) 110 to perform the functions described herein.

In this disclosure, the term “processor” can refer to substantially any computing processing unit or device, including single-core processors, single-processors with software multithreading execution capability, multi-core processors, multi-core processors with software multithreading execution capability, multi-core processors with hardware multithread technology, parallel platforms, and parallel platforms with distributed shared memory. Additionally, a processor can refer to an integrated circuit, an application specific integrated circuit (ASIC), a digital signal processor (DSP), a field programmable gate array (FPGA), a programmable logic controller (PLC), a complex programmable logic device (CPLD), a discrete gate or transistor logic, discrete hardware components, or any combination thereof designed to perform the functions described herein. Further, processors can exploit nano-scale architectures, such as molecular and quantumdot based transistors, switches, and gates, to optimize space usage or enhance performance of user equipment. A processor can also be implemented as a combination of computing processing units.

In some embodiments, the memory 120 includes computer-readable application instructions 150, configured to implement certain embodiments described herein, and a database 150, comprising various data accessible by the application instructions 140. In some embodiments, the application instructions 140 include software elements corresponding to one or more of the various embodiments described herein. For example, application instructions 140 may be implemented in various embodiments using any desired programming language, scripting language, or combination of programming and/or scripting languages (e.g., C, C++, C#, JAVA, JAVASCRIPT, PERL, etc.).

In this disclosure, terms “store,” “storage,” “data store,” data storage,” “database,” and substantially any other information storage component relevant to operation and functionality of a component are utilized to refer to “memory components,” which are entities embodied in a “memory,” or components comprising a memory. Those skilled in the art would appreciate that the memory and/or memory components described herein can be volatile memory, nonvolatile memory, or both volatile and nonvolatile memory. Nonvolatile memory can include, for example, read only memory (ROM), programmable ROM (PROM), electrically programmable ROM (EPROM), electrically erasable ROM (EEPROM), flash memory, or nonvolatile random access memory (RAM) (e.g., ferroelectric RAM (FeRAM). Volatile memory can include, for example, RAM, which can act as external cache memory. The memory and/or memory components of the systems or computer-implemented methods can include the foregoing or other suitable types of memory.

Generally, a computing device will also include, or be operatively coupled to receive data from or transfer data to, or both, one or more mass data storage devices; however, a computing device need not have such devices. The computer readable storage medium (or media) can be a tangible device that can retain and store instructions for use by an instruction execution device. The computer readable storage medium can be, for example, an electronic storage device, a magnetic storage device, an optical storage device, an electromagnetic storage device, a semiconductor storage device, or any suitable combination of the foregoing. A non-exhaustive list of more specific examples of the computer readable storage medium can include: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. In this disclosure, a computer readable storage medium is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

In some embodiments, the steps and actions of the application instructions 140 described herein are embodied directly in hardware, in a software module executed by a processor, or in a combination of the two. A software module may reside in RAM, flash memory, ROM memory, EPROM memory, EEPROM memory, registers, a hard disk, a removable disk, a CD-ROM, or any other form of storage medium known in the art. An exemplary storage medium may be coupled to the processor 110 such that the processor 110 can read information from, and write information to, the storage medium. In the alternative, the storage medium may be integrated into the processor 110. Further, in some embodiments, the processor 110 and the storage medium may reside in an Application Specific Integrated Circuit (ASIC). In the alternative, the processor and the storage medium may reside as discrete components in a computing device. Additionally, in some embodiments, the events or actions of a method or algorithm may reside as one or any combination or set of codes and instructions on a machine-readable medium or computer-readable medium, which may be incorporated into a computer program product.

In some embodiments, the application instructions 140 for carrying out operations of the present disclosure can be assembler instructions, instruction-set-architecture (ISA) instructions, machine instructions, machine dependent instructions, microcode, firmware instructions, state-setting data, configuration data for integrated circuitry, or either source code or object code written in any combination of one or more programming languages, including an object oriented programming language such as Smalltalk, C++, or the like, and procedural programming languages, such as the “C” programming language or similar programming languages. The application instructions 140 can execute entirely on the user’s computer, partly on the user’s computer, as a stand-alone software package, partly on the user’s computer and partly on a remote computer, or entirely on the remote computer or server. In the latter scenario, the remote computer can be connected to the user’s computer through any type of network, including a local area network (LAN) or a wide area network (WAN), or the connection can be made to an external computer (for example, through the Internet using an Internet Service Provider). In some embodiments, electronic circuitry including, for example, programmable logic circuitry, field-programmable gate arrays (FPGA), or programmable logic arrays (PLA) can execute the computer readable program instructions by utilizing state information of the computer readable program instructions to personalize the electronic circuitry, in order to perform aspects of the present disclosure.

In some embodiments, the application instructions 140 can be downloaded to a computing/processing device from a computer readable storage medium, or to an external computer or external storage device via a network 190. A network adapter card or network interface in each computing/processing device receives computer readable program instructions from the network and forwards the computer readable application instructions 140 for storage in a computer readable storage medium within the respective computing/processing device.

In some embodiments, the computer system 100 includes one or more interfaces 160 that allow the computer system 100 to interact with other systems, devices, or computing environments. In some embodiments, the computer system 100 comprises a network interface 165 to communicate with a network 190. In some embodiments, the network interface 165 is configured to allow data to be exchanged between the computer system 100 and other devices attached to the network 190, such as other computer systems, or between nodes of the computer system 100. In various embodiments, the network interface 165 may support communication via wired or wireless general data networks, such as any suitable type of Ethernet network, for example, via telecommunications/telephony networks such as analog voice networks or digital fiber communications networks, via storage area networks such as Fiber Channel SANs, or via any other suitable type of network and/or protocol. Other interfaces include the user interface 170 and the peripheral device interface 175.

In some embodiments, the network 190 corresponds to a local area network (LAN), wide area network (WAN), the Internet, a direct peer-to-peer network (e.g., device to device Wi-Fi, Bluetooth, etc.), and/or an indirect peer-to-peer network (e.g., devices communicating through a server, router, or other network device). The network 190 can comprise copper transmission cables, optical transmission fibers, wireless transmission, routers, firewalls, switches, gateway computers and/or edge servers. The network 190 can represent a single network or multiple networks. In some embodiments, the network 190 used by the various devices of the computer system 100 is selected based on the proximity of the devices to one another or some other factor. For example, when a first user device and second user device are near each other (e.g., within a threshold distance, within direct communication range, etc.), the first user device may exchange data using a direct peer-to-peer network. But when the first user device and the second user device are not near each other, the first user device and the second user device may exchange data using a peer-to-peer network (e.g., the Internet). The Internet refers to the specific collection of networks and routers communicating using an Internet Protocol (“IP”) including higher level protocols, such as Transmission Control Protocol/Internet Protocol (“TCP/IP”) or the Uniform Datagram Packet/Internet Protocol (“UDP/IP”).

Any connection between the components of the system may be associated with a computer-readable medium. For example, if software is transmitted from a website, server, or other remote source using a coaxial cable, fiber optic cable, twisted pair, digital subscriber line (DSL), or wireless technologies such as infrared, radio, and microwave, then the coaxial cable, fiber optic cable, twisted pair, DSL, or wireless technologies such as infrared, radio, and microwave are included in the definition of medium. As used herein, the terms “disk” and “disc” include compact disc (CD), laser disc, optical disc, digital versatile disc (DVD), floppy disk, and Blu-ray disc; in which “disks” usually reproduce data magnetically, and “discs” usually reproduce data optically with lasers. Combinations of the above should also be included within the scope of computer-readable media. In some embodiments, the computer-readable media includes volatile and nonvolatile memory and/or removable and non-removable media implemented in any type of technology for storage of information, such as computer-readable instructions, data structures, program modules, or other data. Such computer-readable media may include RAM, ROM, EEPROM, flash memory or other memory technology, optical storage, solid state storage, magnetic tape, magnetic disk storage, RAID storage systems, storage arrays, network attached storage, storage area networks, cloud storage, or any other medium that can be used to store the desired information and that can be accessed by a computing device. Depending on the configuration of the computing device, the computer-readable media may be a type of computer-readable storage media and/or a tangible non-transitory media to the extent that when mentioned, non-transitory computer-readable media exclude media such as energy, carrier signals, electromagnetic waves, and signals per se.

In some embodiments, the system is world-wide-web (www) based, and the network server is a web server delivering HTML, XML, etc., web pages to the computing devices. In other embodiments, a client-server architecture may be implemented, in which a network server executes enterprise and custom software, exchanging data with custom client applications running on the computing device.

In some embodiments, the system can also be implemented in cloud computing environments. In this context, “cloud computing” refers to a model for enabling ubiquitous, convenient, on-demand network access to a shared pool of configurable computing resources (e.g., networks, servers, storage, applications, and services) that can be rapidly provisioned via virtualization and released with minimal management effort or service provider interaction, and then scaled accordingly. A cloud model can be composed of various characteristics (e.g., on-demand self-service, broad network access, resource pooling, rapid elasticity, measured service, etc.), service models (e.g., Software as a Service (“SaaS”), Platform as a Service (“PaaS”), Infrastructure as a Service (“IaaS”), and deployment models (e.g., private cloud, community cloud, public cloud, hybrid cloud, etc.).

As used herein, the term “add-on” (or “plug-in”) refers to computing instructions configured to extend the functionality of a computer program, where the add-on is developed specifically for the computer program. The term “add-on data” refers to data included with, generated by, or organized by an add-on. Computer programs can include computing instructions, or an application programming interface (API) configured for communication between the computer program and an add-on. For example, a computer program can be configured to look in a specific directory for add-ons developed for the specific computer program. To add an add-on to a computer program, for example, a user can download the add-on from a website and install the add-on in an appropriate directory on the user’s computer.

In some embodiments, the computer system 100 may include a user computing device 145, an administrator computing device 185 and a third-party computing device 195 each in communication via the network 190. The user computing device 145 may be utilized by the primary user select an insurance provider and view the potential drugs suitable for the patient. The administrator computing device 185 is utilized by an administrative user to moderate content and to perform other administrative functions. The third-party computing device 195 may be utilized by third parties to receive communications from the user computing device, transmit communications to the user via the network, and otherwise interact with the various functionalities of the system.

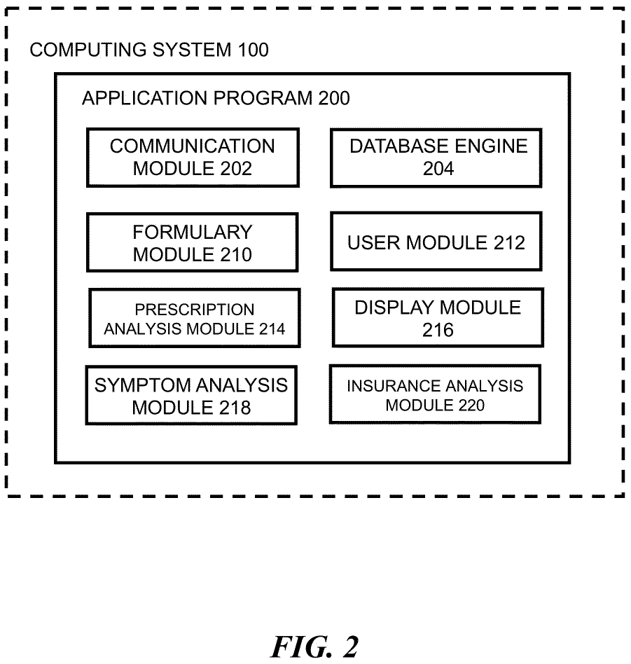

FIG. 2 illustrates an example computer architecture for the application program 200 operated via the computing system 100. The computer system 100 comprises several modules and engines configured to execute the functionalities of the application program 200, and a database engine 204 configured to facilitate how data is stored and managed in one or more databases. In particular, FIG. 2 is a block diagram showing the modules and engines needed to perform specific tasks within the application program 200.

Referring to FIG. 2, the computing system 100 operating the application program 200 comprises one or more modules having the necessary routines and data structures for performing specific tasks, and one or more engines configured to determine how the platform manages and manipulates data. In some embodiments, the application program 200 comprises one or more of a communication module 202, a database engine 204, a formulary module 210, a user module 212, a prescription analysis module 214, display module 216, a symptom analysis module 218, and an insurance analysis module 220.

In some embodiments, the communication module 202 is configured for receiving, processing, and transmitting a user command and/or one or more data streams. In such embodiments, the communication module 202 performs communication functions between various devices, including the user computing device 145, the administrator computing device 185, and a third-party computing device 195. In some embodiments, the communication module 202 is configured to allow one or more users of the system, including a third-party, to communicate with one another. In some embodiments, the communications module 202 is configured to maintain one or more communication sessions with one or more servers, the administrative computing device 185, and/or one or more third-party computing device(s) 195.

In some embodiments, a database engine 204 is configured to facilitate the storage, management, and retrieval of data to and from one or more storage mediums, such as the one or more internal databases described herein. In some embodiments, the database engine 204 is coupled to an external storage system. In some embodiments, the database engine 204 is configured to apply changes to one or more databases. In some embodiments, the database engine 204 comprises a search engine component for searching through thousands of data sources stored in different locations.

In some embodiments, the formulary module 210 provides a means for procuring a formulary and data associated therewith. The formulary module 210 may be in operable communication with the insurance analysis module 220, and prescription analysis module 214 to determine a suitable formulary to display. The formulary module 210 may operate to determine a tier.

In some embodiments, the user module 212 facilitates the creation of a user account for the application system. The user module 212 may allow the user to create a user profile including a company name, username, password, contact information, services offered, OCU’s, social media handles, case templates, signatures, etc.

In some embodiments, the prescription analysis module 214 is in operable communication with the computing device to analyze a prescription and contents thereof. (PLEASE LIST OPTIONS FOR ENTERING THE PRESCRIPTION INFORMATION - I.E., MANUAL ENTRY, OCR, ETC.)

In some embodiments, the display module 216 is configured to display one or more graphic user interfaces, including, e.g., one or more user interfaces, one or more consumer interfaces, one or more video presenter interfaces, etc. In some embodiments, the display module 216 is configured to temporarily generate and display various pieces of information in response to one or more commands or operations. The various pieces of information or data generated and displayed may be transiently generated and displayed, and the displayed content in the display module 216 may be refreshed and replaced with different content upon the receipt of different commands or operations in some embodiments. In such embodiments, the various pieces of information generated and displayed in a display module 216 may not be persistently stored.

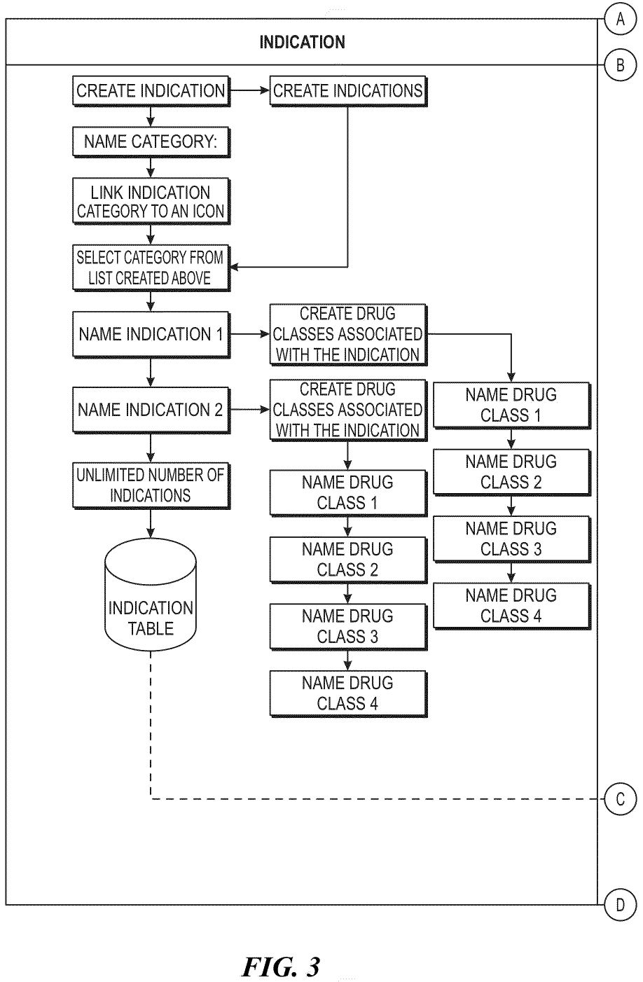

FIG. 3 illustrates a flowchart of the process for creating an indication category, drug container based on the indication and assigning medications to the container, according to some embodiments. An indication category is created including a category name (e.g., cardiology) and an icon link is connected thereto. Indications may be created via a category list (e.g., indication 1 = hypertension) and medication containers are associated to each indication (e.g., container 1 = ACE inhibitor). Each medication may then be assigned to their corresponding containers.

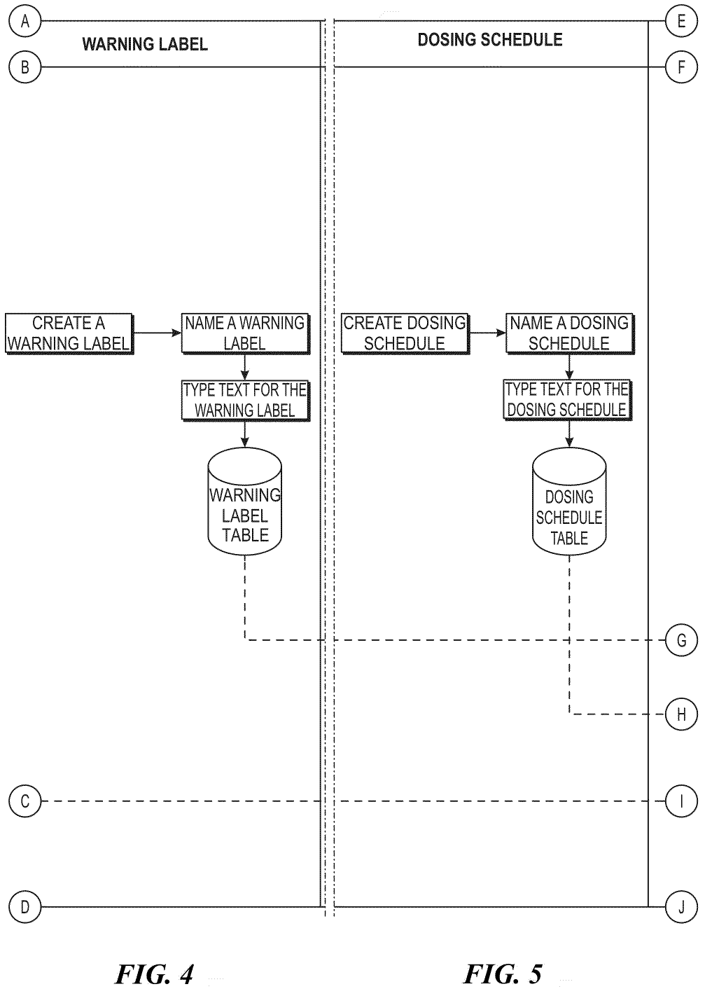

FIG. 4 illustrates a flowchart of a process for generating a warning label. Warning labels are named (e.g., congenital birth defects) and text is entered which may be associated with each warning label. This information may be manually entered or automatically sourced from a third-party database.

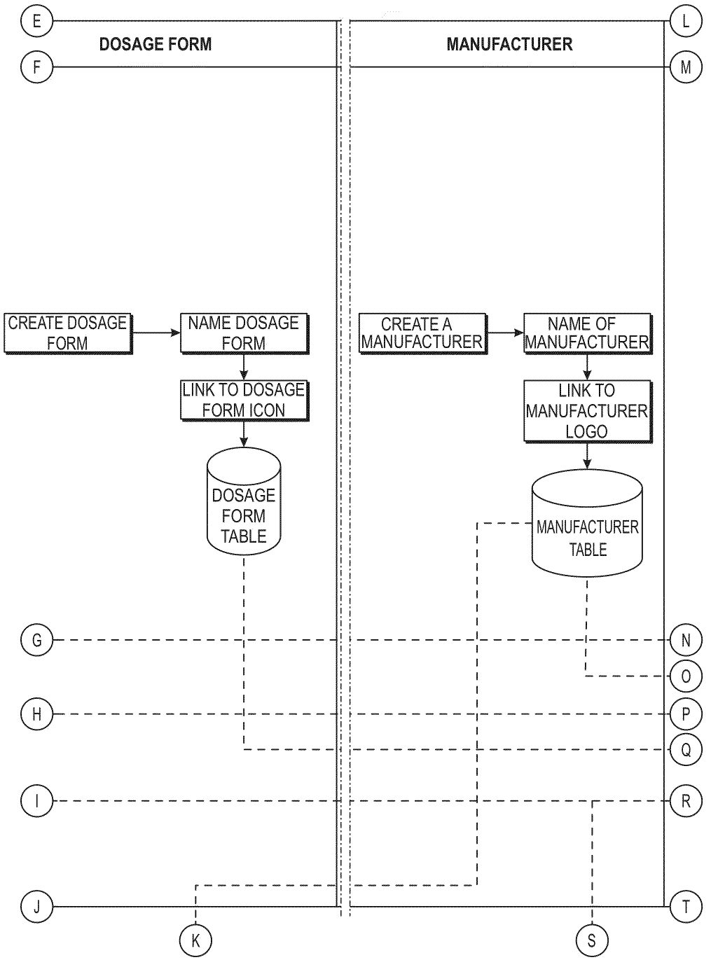

FIG. 5 and FIG. 6 illustrate a flowcharts of the process of generating a dosage schedule provided on a dosage form. The dosage form is named (e.g., oral tablet) and a link to the dosage form is created. Dosing schedules are generated including a dosing schedule name, as well as text to describe the dosing schedule. This information may be automatically sourced from a third-party database.

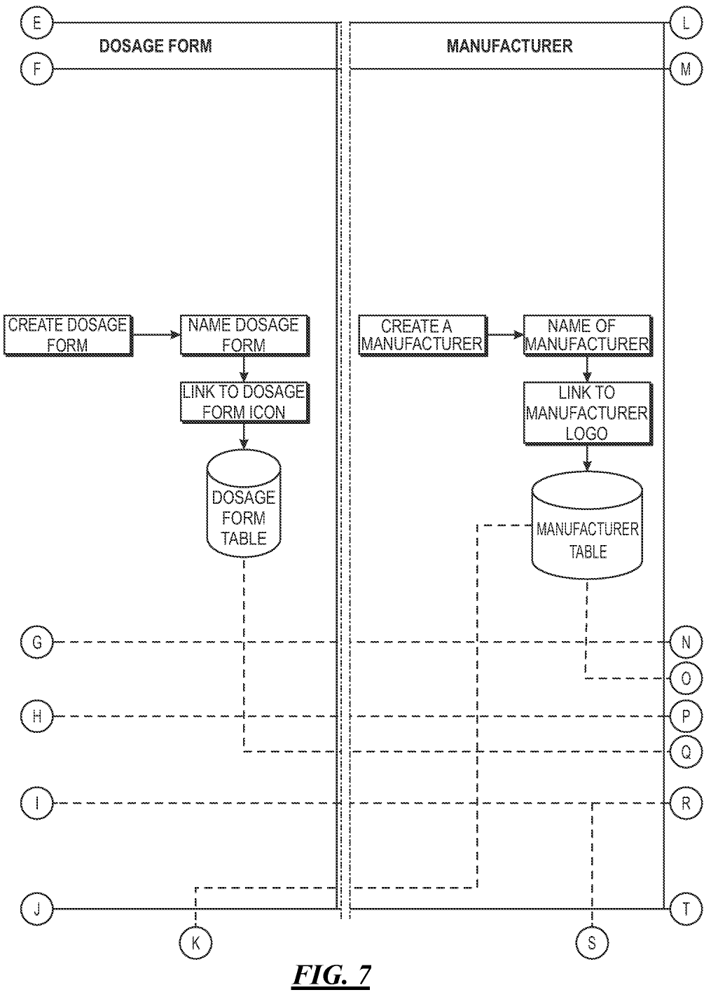

FIG. 7 illustrates a flowchart of the process of generating a manufacturer table. A manufacturer name is entered and a link to the manufacture is generated.

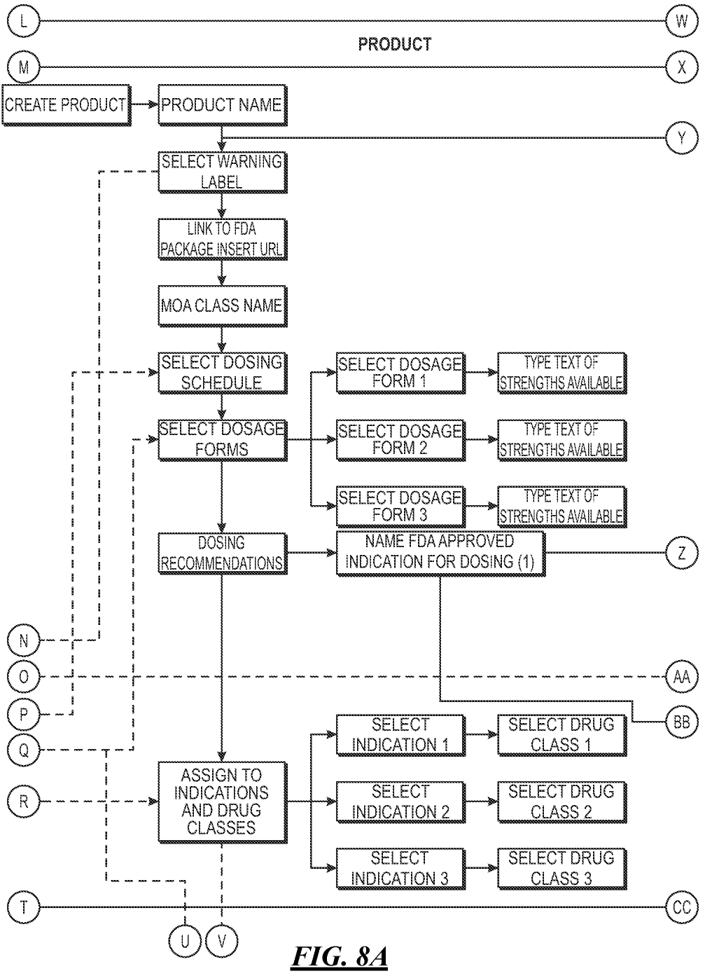

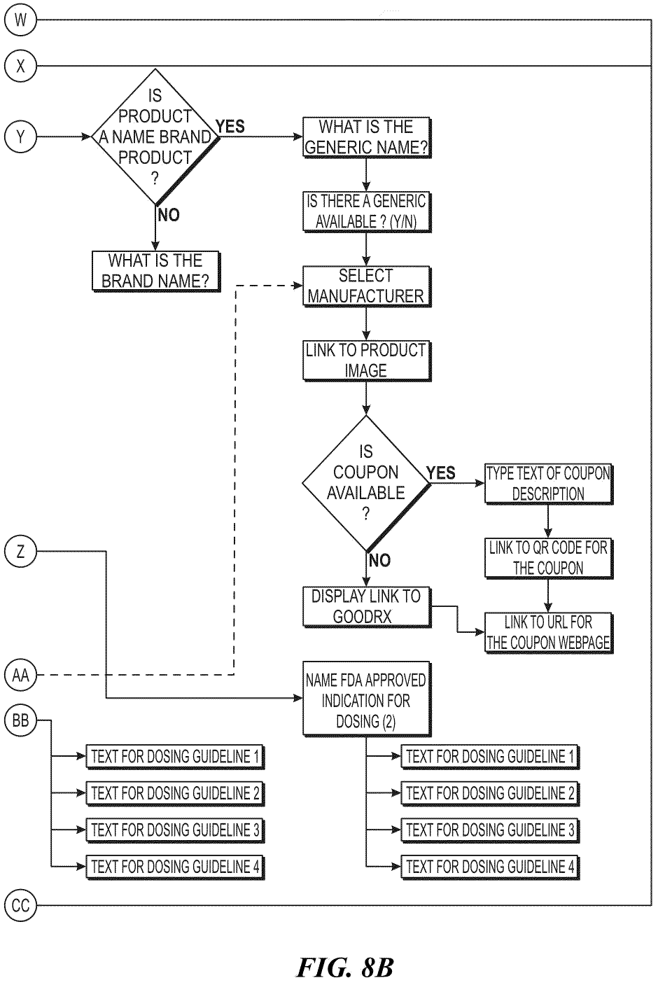

FIG. 8 illustrates a flowchart of the process of distributing medication (i.e., product) information to the user, according to some embodiments. The medication information may include a product name (which may be a brand name and/or generic name of the medication), product logo, product image, available coupons (i.e., discounts, promotions, etc.), warning labels, drug class, dosing regimen (i.e., dosing schedule), dosage forms, recommendations, and the like. This information may then be displayed to the user on the front end interface to allow the user.

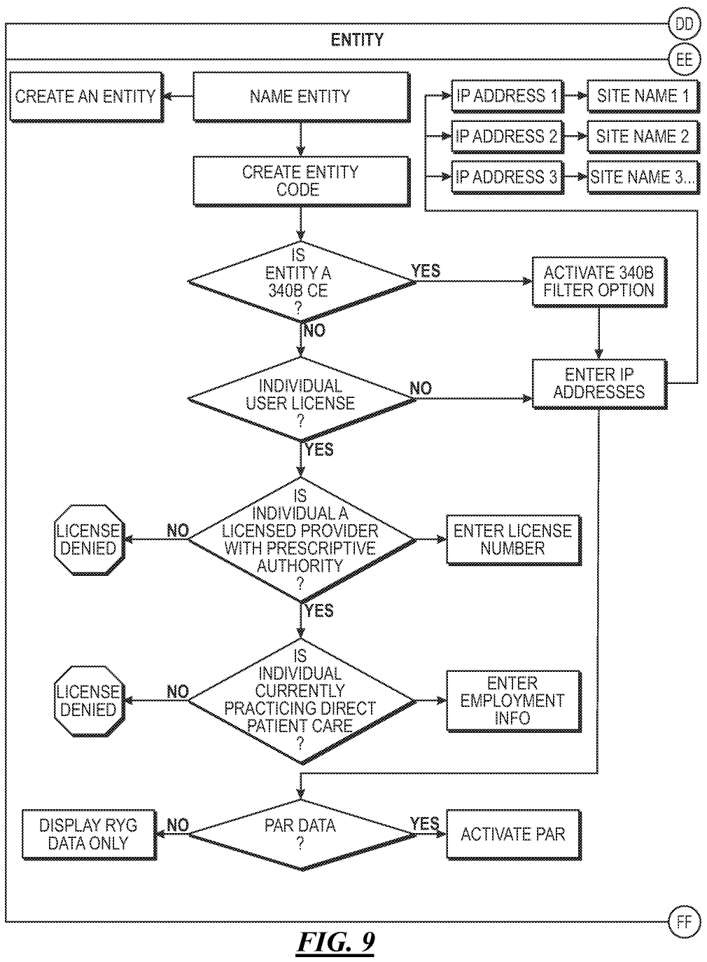

FIG. 9 illustrates a flowchart of the process of determining if the user is a prescriptive authority or if the user is licensed. This process may be performed by an administrative user. In one example, users may select if their entity type, whether they are licensed, and/or if they are a prescriptive authority.

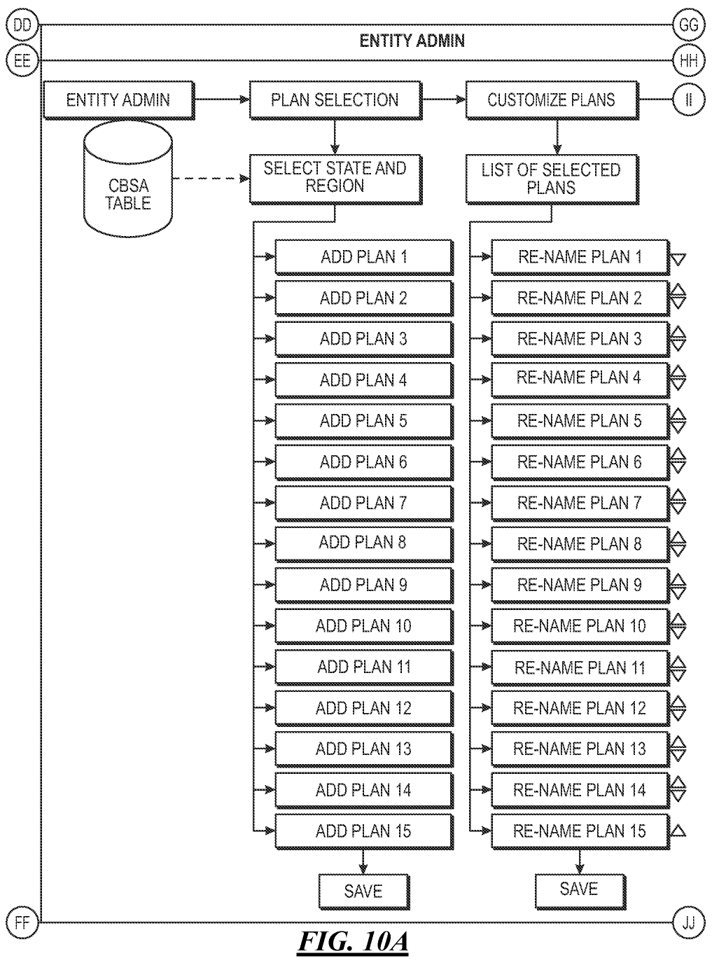

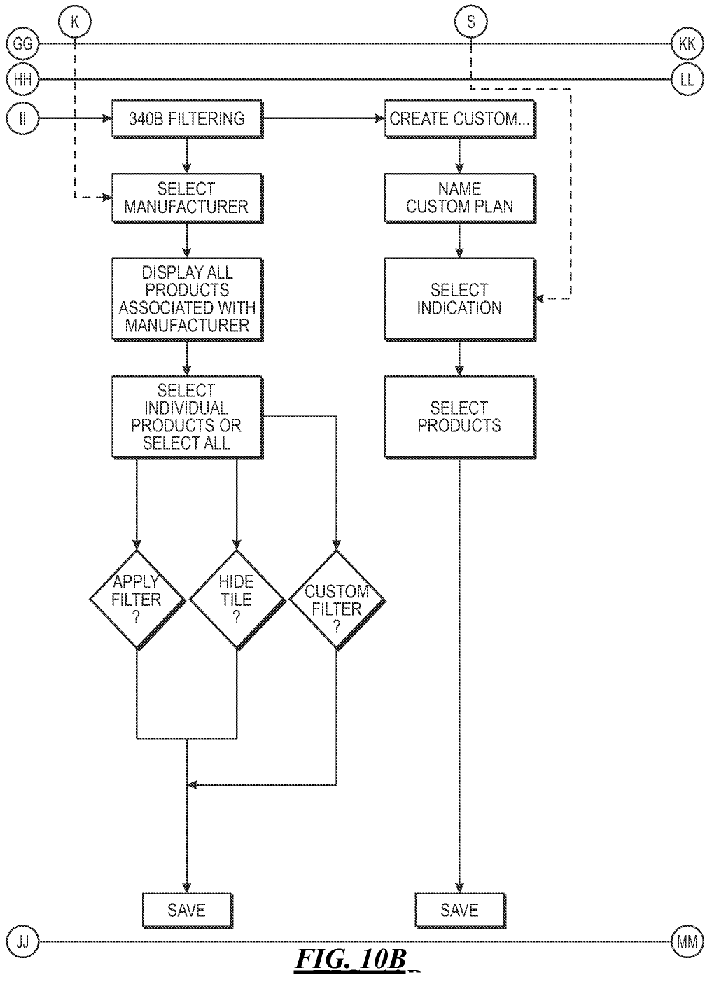

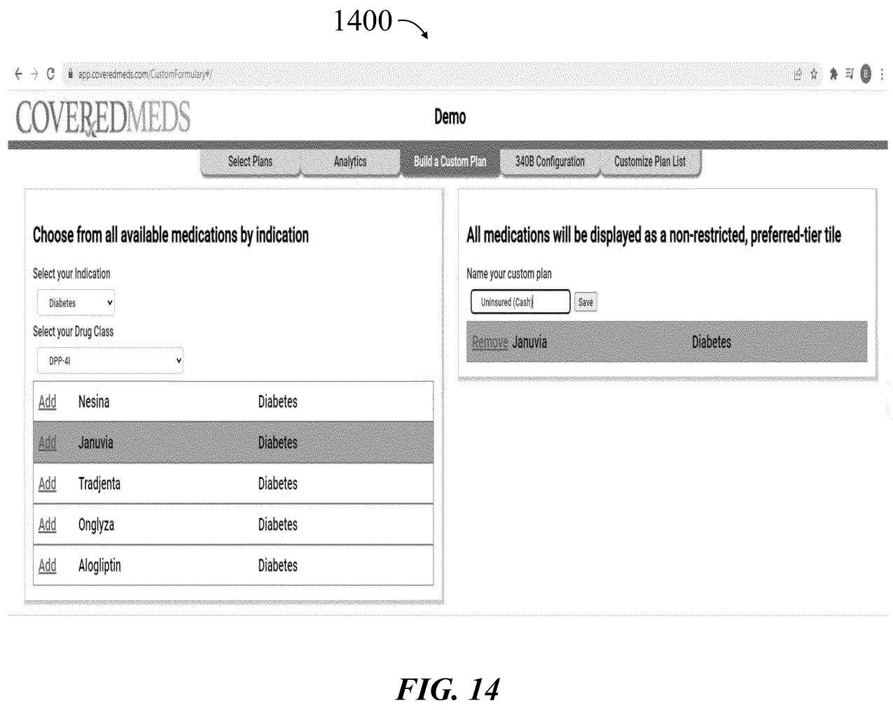

FIG. 10 illustrates a flowchart of the administrative processes performed via the administrative user utilizing the system. This process may include selecting and/or customizing plans which are most common to their practice. To generate the list, the user may select a location upon which plans are displayed based on number of lives covered (e.g., most to least). The user may then select plans to add them to their customized list. The customized list may allow the user to change the names of the plan as well as perform other customization functions (also see FIG. 14).

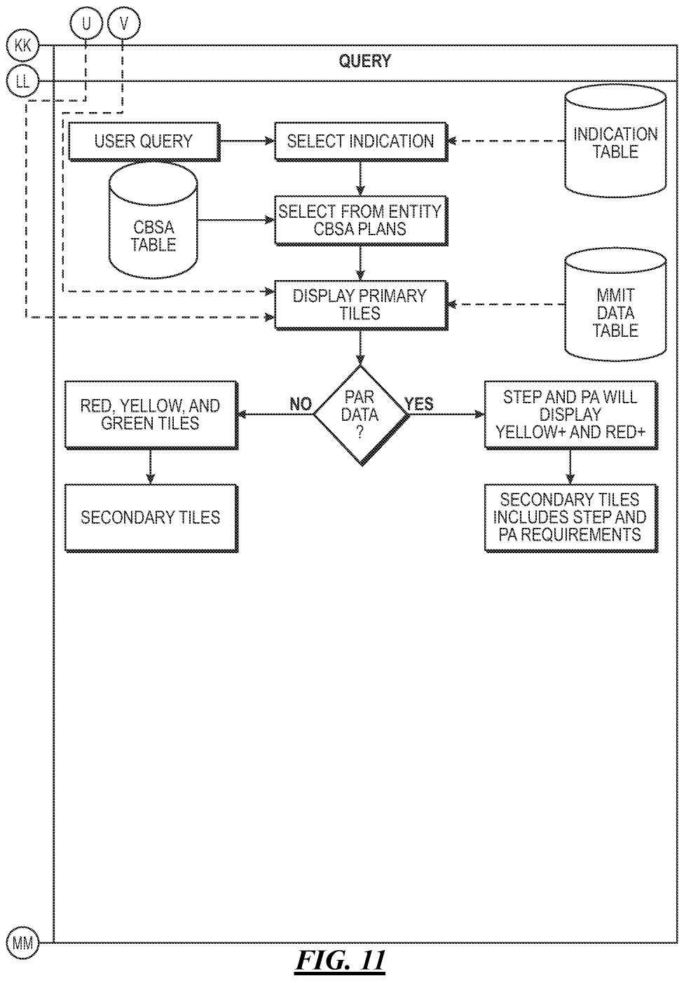

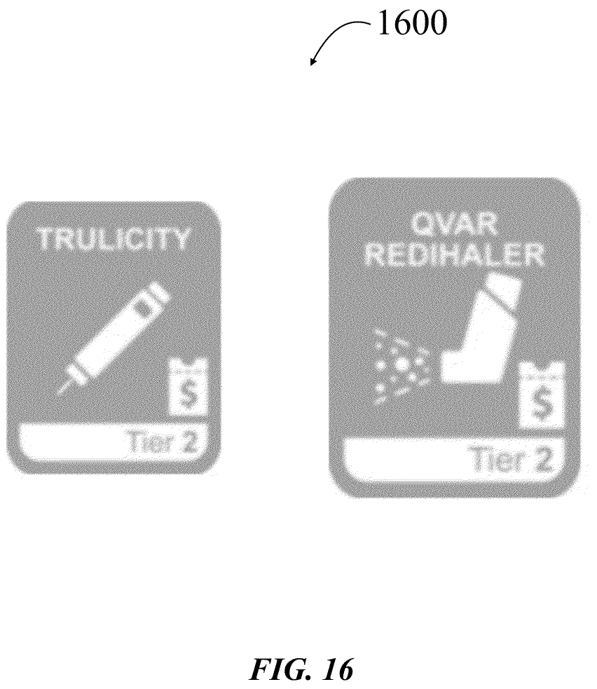

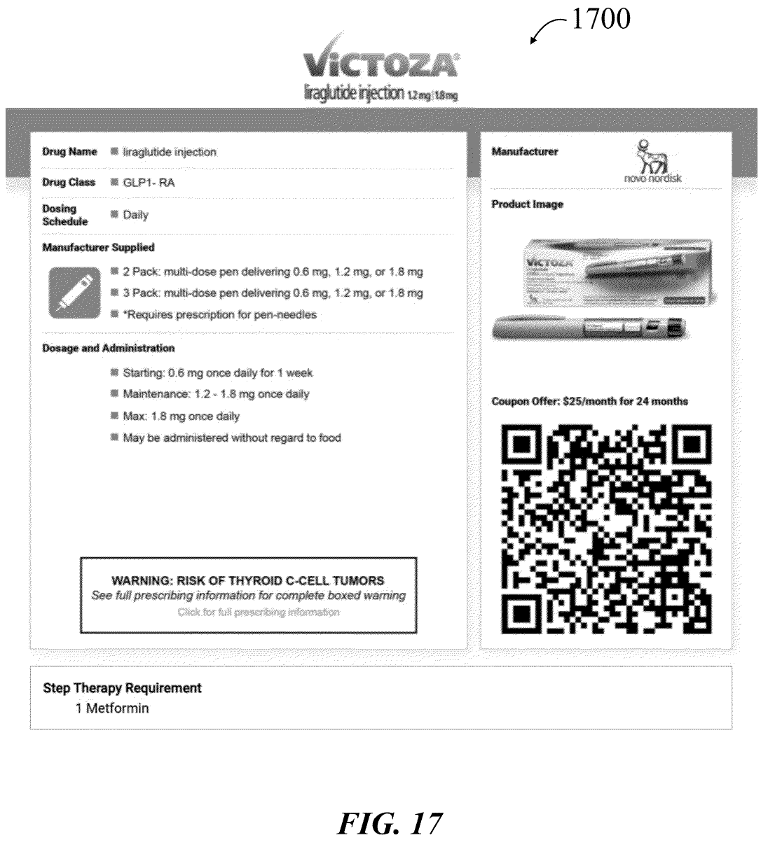

FIG. 11 illustrates a flowchart of a process of the user querying the system to receive medication information. Once queried, various information is shown including the indication, entity plans, MMIT data (displayed on the primary tiles (as shown in FIG. 16 and FIG. 17), PAR data, and the like.

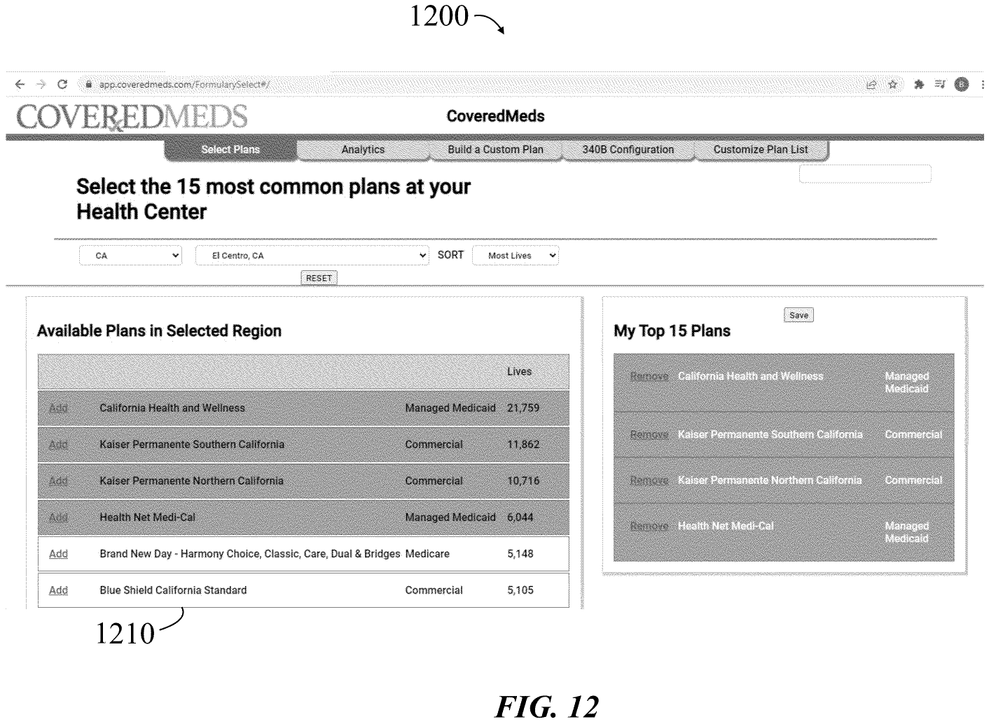

FIG. 12 illustrates a screenshot of the insurance plan selection interface 1200. Users may select their location (e.g., state and city) in a drop down menu. Once the location is selected, the user is displayed a plurality of plans 1210 which they have saved and/or which are available.

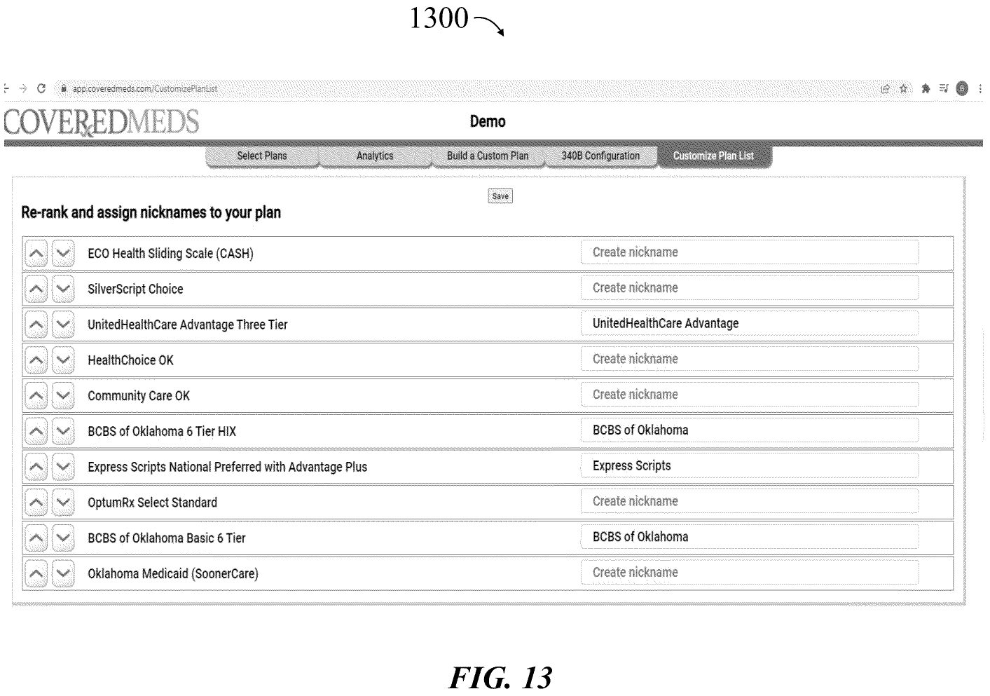

FIG. 13 illustrates a screenshot of the plan customization interface 1300 wherein the user may view a listing of their plans as well as enter a nickname or other customizable information associated with each plan. Further, users may modify the ranking of their plans.

FIG. 14 illustrates a screenshot of the medication selection interface 1400 wherein the user selects from a listing of available medications based on an indication. The user may create filters that will display over any product (i.e., drug) which is displayed on the interface.

In some embodiments, the user may view analytics related to their practice including but not limited to plan metrics, product metrics and the like. In one example, usage statistics are displayed which provide the user with analytics for the number of times each plan has been recalled by a user in their practice (i.e., their entity). Graphical analysis may be provided wherein the x-axis indicated the times an indication has been used and the y-axis indicates months of the year (or other time period).

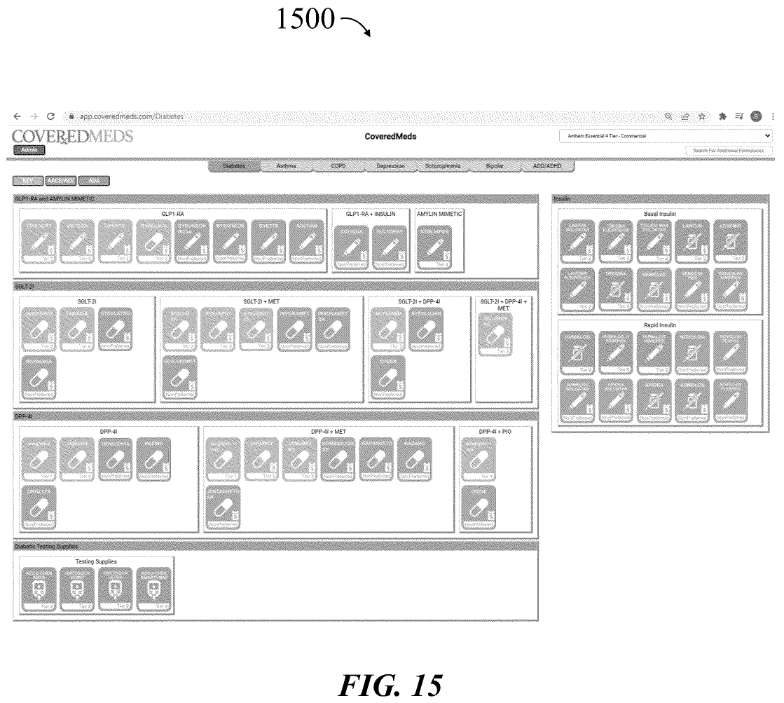

FIG. 15 illustrates a screenshot of an organized interface 1500 showing various medications based on various parameters for each medication. To display the coverage determinations by insurance plan, the clinician first selects the indication for which they are treating their patient from the tabs located at the top of the screen. Once an indication is selected, the user either selects a plan from the drop-down menu in the top right corner of the screen or uses the search for additional formularies function in the top right corner of the screen to word search among all formularies available on the interface. FIG. 16 illustrates a screenshot of the tiles 1600 also shown in FIG. 15. The tiles may be color-coded to indicate insurance coverage. In one example, medications are displayed on a tile that is one of 3 colors. A green tile may indicate that the drug is covered without any restrictions. A yellow tile may indicate that the drug is covered but requires a Step Therapy (i.e., patient must try and fail another product first). A red tile may indicate that the drug is covered by requires a prior authorization (i.e., clinician must submit documentation to the payer that will be reviewed and approved for denied before they will pay for the drug).

FIG. 17 illustrates a screenshot of the medication information interface 1700. The medication information interface may include various details related to the medication including the medication name, class, manufacturer(s), dosage and administration information, warning label, restrictions, coupons, product image(s), and the like.

In this disclosure, the various embodiments are described with reference to the flowchart illustrations and/or block diagrams of methods, apparatus (systems), and computer program products. Those skilled in the art would understand that each block of the flowchart illustrations and/or block diagrams, and combinations of blocks in the flowchart illustrations and/or block diagrams, can be implemented by computer readable program instructions. The computer readable program instructions can be provided to a processor of a general purpose computer, special purpose computer, or other programmable data processing apparatus to produce a machine, such that the instructions, which execute via the processor of the computer or other programmable data processing apparatus, create means for implementing the functions or acts specified in the flowchart and/or block diagram block or blocks. The computer readable program instructions can be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks. The computer readable program instructions can be loaded onto a computer, other programmable data processing apparatus, or other device to cause a series of operational acts to be performed on the computer, other programmable apparatus, or other device to produce a computer implemented process, such that the instructions that execute on the computer, other programmable apparatus, or other device implement the functions or acts specified in the flowchart and/or block diagram block or blocks.

In this disclosure, the block diagrams in the Figures illustrate the architecture, functionality, and operation of possible implementations of systems, methods, and computer program products according to the various embodiments. Each block in the flowchart or block diagrams can represent a module, segment, or portion of instructions, which comprises one or more executable instructions for implementing the specified logical function(s). In some embodiments, the functions noted in the blocks can occur out of the order noted in the Figures. For example, two blocks shown in succession can, in fact, be executed concurrently or substantially concurrently, or the blocks can sometimes be executed in the reverse order, depending upon the functionality involved. In some embodiments, each block of the block diagrams and/or flowchart illustration, and combinations of blocks in the block diagrams and/or flowchart illustration, can be implemented by a special purpose hardware-based system that performs the specified functions or acts or carry out combinations of special purpose hardware and computer instructions.

In this disclosure, the subject matter has been described in the general context of computer-executable instructions of a computer program product running on a computer or computers, and those skilled in the art would recognize that this disclosure can be implemented in combination with other program modules. Generally, program modules include routines, programs, components, data structures, etc. that perform particular tasks and/or implement particular abstract data types. Those skilled in the art would appreciate that the computer-implemented methods disclosed herein can be practiced with other computer system configurations, including single-processor or multiprocessor computer systems, mini-computing devices, mainframe computers, as well as computers, hand-held computing devices (e.g., PDA, phone), microprocessor-based or programmable consumer or industrial electronics, and the like. The illustrated embodiments can be practiced in distributed computing environments where tasks are performed by remote processing devices that are linked through a communications network. Some embodiments of this disclosure can be practiced on a stand-alone computer. In a distributed computing environment, program modules can be located in both local and remote memory storage devices.

In this disclosure, the terms “component,” “system,” “platform,” “interface,” and the like, can refer to and/or include a computer-related entity or an entity related to an operational machine with one or more specific functionalities. The disclosed entities can be hardware, a combination of hardware and software, software, or software in execution. For example, a component can be a process running on a processor, a processor, an object, an executable, a thread of execution, a program, and/or a computer. By way of illustration, both an application running on a server and the server can be a component. One or more components can reside within a process and/or thread of execution and a component can be localized on one computer and/or distributed between two or more computers. In another example, respective components can execute from various computer readable media having various data structures stored thereon. The components can communicate via local and/or remote processes such as in accordance with a signal having one or more data packets (e.g., data from one component interacting with another component in a local system, distributed system, and/or across a network such as the Internet with other systems via the signal). As another example, a component can be an apparatus with specific functionality provided by mechanical parts operated by electric or electronic circuitry, which is operated by a software or firmware application executed by a processor. In such a case, the processor can be internal or external to the apparatus and can execute at least a part of the software or firmware application. As another example, a component can be an apparatus that provides specific functionality through electronic components without mechanical parts, wherein the electronic components can include a processor or other means to execute software or firmware that confers at least in part the functionality of the electronic components. In some embodiments, a component can emulate an electronic component via a virtual machine, e.g., within a cloud computing system.

The phrase “application” as is used herein means software other than the operating system, such as Word processors, database managers, Internet browsers and the like. Each application generally has its own user interface, which allows a user to interact with a particular program. The user interface for most operating systems and applications is a graphical user interface (GUI), which uses graphical screen elements, such as windows (which are used to separate the screen into distinct work areas), icons (which are small images that represent computer resources, such as files), pull-down menus (which give a user a list of options), scroll bars (which allow a user to move up and down a window) and buttons (which can be “pushed” with a click of a mouse). A wide variety of applications is known to those in the art.

The phrases “Application Program Interface” and API as are used herein mean a set of commands, functions and/or protocols that computer programmers can use when building software for a specific operating system. The API allows programmers to use predefined functions to interact with an operating system, instead of writing them from scratch. Common computer operating systems, including Windows, Unix, and the Mac OS, usually provide an API for programmers. An API is also used by hardware devices that run software programs. The API generally makes a programmer’s job easier, and it also benefits the end user since it generally ensures that all programs using the same API will have a similar user interface.

The phrase “central processing unit” as is used herein means a computer hardware component that executes individual commands of a computer software program. It reads program instructions from a main or secondary memory, and then executes the instructions one at a time until the program ends. During execution, the program may display information to an output device such as a monitor.

The term “execute” as is used herein in connection with a computer, console, server system or the like means to run, use, operate or carry out an instruction, code, software, program and/or the like.

In this disclosure, the descriptions of the various embodiments have been presented for purposes of illustration and are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein. Thus, the appended claims should be construed broadly, to include other variants and embodiments, which may be made by those skilled in the art.

What is claimed is:

1. A system for determining suitable medications for a patient based on the patient’s insurance provider, the system comprising:
at least one user computing device in operable connection with a user network;
an application server in operable communication with the user network, the application server configured to host an application system for providing a plurality of medication information, the application system having a user interface for providing access to the application system through the user computing device;
a plurality of tiles displayed on the user interface, wherein each tile is associated with a medication.

2. The system of claim 1, wherein the plurality of medication information includes at least one of the following: a medication name, a medication class, a dosing schedule, a dosage recommendation, a warning label, a coupon, a manufacturer, and a product image.

3. The system of claim 1, wherein each of the plurality of tiles are color-coded.

4. The system of claim 3, wherein each of the color-coded tiles indicate a restriction status on the medication associated thereto.

5. The system of claim 1, wherein each of the color-coded tiles displays a dosage form associated with the medication associated thereto.

6. The system of claim 1, wherein each of the color-coded tiles displays a copay tier associated with the medication associated thereto.

7. The system of claim 1, wherein each of the color-coded tiles displays a coupon availability associated with the medication associated thereto.

8. The system of claim 1, further comprising a plan customization interface permitting the user to select from one or more plans and rank each of the one or more plans.

9. The system of claim 8, wherein the plan customization interface permits the user to select a customized name for each plan.

10. The system of claim 1, further comprising an analytics interface to permit the user to view analytics associated with an entity.

11. A method for displaying suitable medications for a patient based on the patient’s insurance provider, the system comprising:
selecting an insurance provider with which a patient has a policy;
selecting at least one indication category;
selecting at least one indication;
displaying one or more medications based on the at least one selected indication,
wherein the at least one medication is displayed on a color-coded tile.

12. The method of claim 11, wherein a plurality of medication information is displayed, wherein the plurality of medication information includes at least one of the following:
a medication name, a medication class, a dosing schedule, a dosage recommendation,
a warning label, a coupon, a manufacturer, and a product image.

13. The method of claim 11, wherein each of the color-coded tiles indicate a restriction status on the medication associated thereto.

14. The method of claim 11, wherein each of the color-coded tiles displays a dosage form associated with the medication associated thereto.

15. The method of claim 11, wherein each of the color-coded tiles displays a copay tier associated with the medication associated thereto.

16. The method of claim 11, wherein each of the color-coded tiles displays a coupon availability associated with the medication associated thereto.

17. The method of claim 11, further comprising a plan customization interface permitting the user to select from one or more plans and rank each of the one or more plans.

18. The method of claim 17, wherein the plan customization interface permits the user to select a customized name for each plan.

19. The method of claim 11, further comprising an analytics interface to permit the user to view analytics associated with an entity.
